(19) United States
(12) Patent Application Publication
(10) Pub. No.: US 2020/0305795 A1
(43) Pub. Date: Oct. 1, 2020

(54) VIRTUAL ELECTRODE TEMPLATE SYSTEM FOR NEUROLOGICAL MONITORING

(71) Applicant: RHYTHMLINK INTERNATIONAL, LLC, Columbia, SC (US)

(72) Inventors: Harrison Floyd, Columbia, SC (US); Gabriel ORSINGER, Columbia, SC (US)

(21) Appl. No.: 16/902,804

(22) Filed: Jun. 16, 2020

Related U.S. Application Data
(63) Continuation of application No. PCT/US2019/026726, filed on Apr. 10, 2019.
(60) Provisional application No. 62/655,381, filed on Apr. 10, 2018.

(51) Int. Cl. A61B 5/00; G06T 7/73; A61B 5/107; A61B 5/04

(52) U.S. Cl. CPC A61B 5/6841; A61B 5/742; A61B 5/7475; G06T 7/73; A61B 5/6867; A61B 5/1072; A61B 5/04001; A61B 5/7425; A61B 5/6814

(57) ABSTRACT

A method for placing electrodes on a patient uses a mobile device with built-in camera to generate a customized, virtual electrode template for neurological monitoring. The camera captures a digital image of the patient and is programmed to find landmarks, use analysis, and respond to manual input for electrode locations and generate a virtual electrode template. Facial recognition and edge detection software determines the outline of the patient's head when in the field of view of the camera monitor. The user then may assist the camera in identifying landmarks on the patient's head, such as the nasion, the inion, and the pre-auricular points, to facilitate generation of the virtual electrode template on the image in the camera's viewfinder, which template moves and rotates with the patient. The virtual electrode template guides the user in establishing a set of electrodes on the patient's head for neurological monitoring.

### CROSS-REFERENCE TO RELATED APPLICATIONS

This application claims the benefit of International Application PCT/US19/26726 filed Apr. 10, 2019, which claims priority to U.S. Provisional Application No. 62/655,381, filed Apr. 10, 2018.

### TECHNOLOGY FIELD

The disclosure relates generally to neurological monitoring and, more specifically, to the task of placing electrodes in locations on a patient's scalp for neurological monitoring.

### BACKGROUND

To perform neurological monitoring, electrodes are attached to the head of a patient. This process may be performed by a trained technician in a hospital or research setting where the technician can carefully place the electrodes in the correct locations. However, at other times, neither a skilled technician nor the time for careful placement is not available. Emergency medical service technicians may have thorough training in first aid and general emergency medical care, they may not be trained in electrode placement for neurological monitoring, especially under emergency circumstances when access to assistance is not possible. While there is latitude in the precise positions of the electrodes, depending on circumstances and the nature of the medical issue of concern, it is nonetheless important to place the electrodes in the correct locations on the scalp of the patient.

### SUMMARY

This disclosure describes a system and method to enable, for example, an emergency medical technician, or those with limited training, to quickly and accurately establish the scalp locations for electrodes and placing the electrodes on those scalp locations of a patient by someone who may have limited training.

By establishing the scalp locations for electrode positions, it is meant that a set of locations on the scalp of the patient have been identified as those where electrodes are to be positioned. The position of an electrode is wherever it is placed; the location of the electrode is where the electrode is intended to be placed.

The disclosure further describes a process of identification of locations for electrode positions using a computer interface that includes a monitor, a display or camera viewfinder, a camera or video camera operable to capture an image, and a computer processor with associated computer memory, which are interconnected. The term “capture” or “capturing” an image means to acquire a digital file that can, when displayed, reproduces the image. The processor is programmed to assist a user to know the location on a patient's scalp for positioning and attaching neurological monitoring electrodes. The present method may be implemented in a mobile device, such as a smart phone, tablet or pad computer, or laptop programmed using a software application.

The present software program is designed to recognize an image of a human face or head in a digital image captured by a camera. The software determines the presence and orientation of the patient's head, assisted by landmarks, which term is used here to mean readily detectable features on a human head, marks made on, or markers placed on the patient's head specifically to facilitate detection of a human head and its orientation. The software then proceeds to generate a virtual electrode template superimposed on the captured digital image of the patient's head, which captured digital image appears to move as, and to the extent that, the image of the patient's head moves. The software aligns digital images of electrodes with the virtual electrode template, which extends over and around the patient's head, and shows where electrodes are to be positioned and attached, with the virtual (digital) electrode template simulating a physical electrode template to serve as a guide for electrode placement in the same manner as a physical template.

The landmarks on the patient's head may be, for example, the nasion, the inion, and pre-auricular points. Other landmarks may also be used, or created ad hoc using an object temporarily placed on the patient or a mark made on the patient's head. Relationships among these landmarks enable other electrode positions to be determined, perhaps by proximity or by calculations between adjacent landmark-associated electrode positions, for any additional number of electrodes, including the twenty electrodes on the scalp in the case of neurological monitoring using the 10-20 International Standard set of electrodes for neurological monitoring.

Electrodes may be applied to the patient's head in accordance with the instructions that are appropriate for the particular electrodes, including using adhesives, inserting the electrodes into the scalp of the patient, or launching the electrodes from a holder so that the electrodes embed themselves in the surface of the patient's scalp.

From input via the user interface, such as the touch of a user to an image of a landmark appearing on a touch-screen monitor with touch-screen capability, the user verifies that a location on the image is a landmark. The computer processor is able to tag that location in the virtual image as the position for one electrode. Additional landmarks may be similarly located and positions for electrodes may be established by their relationships with respect to landmarks and to other electrodes. From these landmarks and markers, the software used to program the computer processor generates the virtual electrode template and associates it with the image of the patient's head. Thus, the image of the patient's head on the monitor, the display or the camera view finder shows the image captured by the camera and, superimposed and aligned therewith, that the virtual electrode template, oriented as it would be were a real electrode template physically on the patient's head. This virtual electrode template tracks the movement of the patient's head, moving with the patient's head as the patient moves.

The disclosure describes a system that recognizes the human head and body. The system recognizes body features such as the head, the nose, eyes and ears and can discern the orientation of the human head from body features. The system may use image-recognition software to discern so-called “landmarks” such as the nasion, inion, left and right auricules and pre- and post-auricular points. In another example, markers may be applied to the human body to create temporary features of that body. For example, reflective stickers, that allow the system to find, and to discern the orientation of, and to track the movement of the patient using digital images produced by the system's digital camera and its processor appropriately programmed for detecting edges and recognizing faces.

The disclosure describes a method for neurological monitoring including the steps of: capturing a digital image of a head; recognizing at least two body features of the head in the digital image; determining locations from the digital image at which electrodes are to be placed; generating a virtual electrode template from those locations that shows the locations for electrode positions from the features; and placing the electrodes at those positions.

The disclosure describes another method for neurological monitoring including the steps of: identifying reference points on a patient's scalp, obtaining patient measurements, obtaining patient images, calculating target locations, generating a three-dimensional virtual electrode template, identifying electrodes by comparing positions of identified electrodes to calculated target locations, guiding a user to apply identified electrodes to target locations, comparing final electrode positions to calculated target locations, generating and transmitting a report for approval, and notifying the user of set up approval.

One aspect of the disclosure is the use of a smart phone with its existing high-resolution camera, which may include facial recognition capability to automatically identify and focus on the face of an individual in the field of view. The smart phone may also include touch screen input technology so that finger gestures, such as swiping with the finger across the smart phone display, can be used with the graphical user interface to facilitate input to the software by, for example, identifying to the computer processor of the smart phone the locations of the landmarks, markers, or input related to other features of the patient's head.

Another aspect of the disclosure is a software application that recognizes a patient's head and, perhaps assisted by landmarks and markers, determines the orientation of the head, and then generates a virtual electrode template onto an image of the head of the patient. The software application may be downloaded to a computer processor for providing facial recognition and boundary-recognition software to identify the shape and orientation of the head of a person in the camera's field of view. The software can then generate the virtual electrode template for that patient's head and “lock” it onto the image of the head of the patient, as that image appears in the field of view, so that, as the person moves and the image reflects those movements, the virtual electrode template will appear to move together with the moving image of the person. To lock the virtual electrode template onto the patient's head means that the image of the template tracks the image of the patient's head in translation and rotation to give the appearance that the template is actually on the head of the patient appearing in the camera's field of view.

Another aspect of the disclosure is that the template may be generated by software programmed to find—or to accept input identifying—certain features of the patient's head that are landmarks or otherwise guides to the shape and orientation of the patient's head and which therefore help to find the locations for the electrodes that are to be used in neurological monitoring. The top of the head, the eyes, the ears, the nose and the chin are features of a head that are readily perceived, even from a distance. The pre-auricular points, for example, are easily located with respect to the ears and ear canal. The nasion is easily located from the nose in profile. The software may, for example, be able to find the nasion, the anion, and the pre-auricular points and plot the virtual electrode template based on those landmarks. Alternatively, these landmarks and other places on the head of a patient may be marked or highlighted by placing small markers on the patient's head that the camera is programmed to detect, locate and relate to each other in order to establish and confirm the orientation of the patient's head.

Yet another alternative to initiating the virtual template is for the user to input, by touching the touch-sensitive view screen of the smart phone or display of a computer programmed with touch screen technology, the locations on the image of the patient's head where the nasion, anion and pre-auricular points appear when the patient is in the field of view. The programmed computer processor then accepts as input those locations indicated by the touch of the user and proceeds to generate the balance of the virtual electrode template of locations for electrode positions for neurological electrode monitoring based on them. By touching the image features on the digital image shown on the viewfinder of a camera or monitor of a computer display, image features may be identified to the processor.

Another aspect of the disclosure is that the patient can move with respect to the camera and the computer processor can be programmed to move and rotate the virtual electrode template in accordance with the patient's movements so that the user can see all sides of the template and the locations of the electrode positions identified on it appearing to remain superimposed on the image of the patient's head appearing to turn and move together with the movement of the patient, visible in the computer monitor or camera viewer, as long as the patient is in the field of view of the camera, no matter which direction the patient is facing. Therefore, the user can place the electrodes of a complete set on the sides of the patient's head using the virtual electrode template as a physical template, guide or map, merely by rotating the camera with respect to the patient until all electrodes have been attached to the patient's head at the electrode locations as guided by the virtual template.

Another aspect of the disclosure is that the electrodes are recognized in real time. This aspect can help guide the placement of the electrodes, or to adjust the positions of electrodes so they are placed in the right locations.

An aspect of the disclosure includes the use of electrodes that are recognized in real time combined with a system that can identify electrode application sites. This aspect allows the system to compare the actual positions of the electrodes with the proper application locations to confirm the correct placement of the electrodes and to guide in the adjustment when required to achieve the correct placement of the electrodes.

Those skilled in electrode placement for neurological and cardiovascular monitoring will appreciate these and other aspects of the disclosure and their advantages from a careful reading of the Detailed Description below, accompanied by the drawings

### BRIEF DESCRIPTION OF THE DRAWINGS

In the figures,

FIG. 1 is a profile of a patient and a smart phone showing the image of the patient on the screen, according to an aspect of the disclosure;

FIG. 2 is again a profile of the patient and a smart phone showing the patient's image as in FIG. 1, now with several locations for electrode positions identified and several electrodes in position at those locations, according to an aspect of the disclosure;

FIG. 3 is a third image of the patient with more electrodes in place and, in the monitor of the smart phone, the image is rotated with a finger swipe to present new locations where electrodes are to be positioned and attached;

FIG. 4 is a process flow diagram according to an aspect of the present method;

FIG. 5 is a fourth image of a patient with an operator wearing a visual device that guides the operator in marking the patient for neurological monitoring; and

FIG. 6 is a process flow diagram according to an aspect of the present method.

### DETAILED DESCRIPTION

Herein is disclosed a method and system for positioning electrodes on the body of a person for neurological monitoring or other types of electrode monitoring such as cardiac monitoring. Neurological monitoring involves positioning, that is, placing a set of electrodes in specific positions on the head of the patient and either for receiving signals through electrodes from the brain or stimulating the brain through the electrodes.

To perform neurological or cardiovascular monitoring, electrodes must be attached to the patient. Accurate and complete electrode placement of a set of electrodes facilitates acquisition of useful information about the patient's brain. In neurological monitoring, there are locations prescribed by standards-setting organizations for the specific numbers and specific locations of electrodes that may be used.

According to the present method for neurological monitoring, for example, the method includes capturing a digital image of the head of a person from all sides and the top, using a camera, such as a digital camera and, if convenient, a digital video camera. An image is “captured” when it is stored in a digital memory device. A computer processor is programmed to identify the the image of the human head stored in that captured digital image by identifying features of the image of the head in the digital image in order to determine that the image includes a head and the orientation of the head. Features are the equivalent of landmarks on terrain that are guides in determining, in some cases, what terrain it is. Features that help to determine whether an image includes a human head, for example, include the nose, eyes, ears, and chin.

The present method for positioning electrodes uses a mobile device with a camera, a viewer, a processor, available memory, and a user interface. The mobile device may also have “touch-screen technology” which means the user interface includes the ability to interpret finger gestures and finger touches as input, including user selections and simple instructions from the user such as to insert here on the monitor. The mobile device may be a dedicated device or a device ordinarily used for other purposes but having the aforementioned capabilities, such as cell phone or a small computer such as a palm top or laptop computer programmed with a computer application for effecting the additional capabilities of the present disclosure.

Software that has programmed the handheld device's processor is used to identify features in a captured digital image that may include features characteristic of a human head that can be used to identify that a human head appears in the image and its orientation. Such software is widely referred to as “facial recognition software” and is known for use in not only recognizing a human face but in identifying a human face. In the present disclosure, such technology need only recognize a human head, its orientation, and a few features such as the nose, the bridge of the nose, and the ears. Other features may be helpful in the present application, such as the top of the head, the ear canals, and the bump on the back of the head.

Using the locations of those features in the digital image of the head of the person, a virtual template is developed and associated with that digital image. A virtual template is developed mathematically using the spatial relationships among the features, just as a physical template would be physically constructed. A physical template would be constructed by a person trained in applying certain well-known protocols in determining the number and locations for positions of physical electrodes based spatial relationships among the features of the patient's head and the relationships among those features. The software programming would generate a set of locations for electrode positions from the locations of features in the digital image using the same protocols and may place marks on the digital image, as if it were a map with marks on it to indicate locations where electrodes are to be placed on the patient's head. The template so constructed may be displayed on the monitor of a small computer or the camera viewer of the mobile device with the relative locations of the electrodes on the image of the human head visible to specify the electrode positions thereby serving as a guide for the user in placing electrodes on the human head.

The user then proceeds to attach a set of electrodes to locations on said patient that correspond to the marks on the virtual electrode template displayed on the monitor.

Features may be recognized by software using edge detection and pattern detection, or recognition may be simplified or augmented by markers placed on the patient that, like a lighthouse on a coast, increase the speed and accuracy of the computer processor in recognizing those areas of the digital image of the patient.

Additionally or alternatively, the computer processor may be programmed to accept input from the user as to the locations of features in the digital image, such as the touch of the user's finger or a stylus on a touch-sensitive monitor pointing to a location on the digital image where the user has identified a feature at that location in that digital image.

The digital image is thus used by software as a surrogate for the head of the patient. A virtual electrode template may be created for an image of a human head using the digital image. Such a virtual template may be facilitated by any of several techniques for positioning images of electrodes (virtual electrodes) on the virtual electrode template that is superimposed on that digital image of the patient's head. Positioning an image of an electrode means that locations are identified in the image for electrodes, thus generating a virtual template, and so marked on the image as electrode positions to guide the user in identifying the locations on the patient's head where electrodes are to be attached, in accordance with the virtual template.

Moreover, the computer processor may confirm on the virtual template that the physical electrodes are correctly placed with respect to the position of the virtual electrode locations as each of the real electrodes is applied and appears on the refreshed digital image, and assists the user in making adjustments in the electrode positions if the real electrodes are not quite in the proper positions.

The computer processor may generate locations for electrode positions from the landmarks it finds. For example, if the nasion, inion, and the auricles are used as digital image features, the computer process can determine the locations for positions of electrodes from those features using the same, well-known analysis a trained technician uses with respect to those features, namely, calculating the locations for positions based on these landmarks.

After the virtual electrode template is generated, the user then proceeds to attach electrodes to the patient as guided by the virtual electrode template. The computer processor confirms the locations of the electrodes or guides the user in adjusting the position of an errant electrode to the appropriate location. The computer processor may highlight an image of an errant electrode to urge the technician to adjust its position. The computer processor may confirm the placement of electrodes when the image of the position of the electrode corresponds to the position indicated for that electrode by the virtual electrode template. A location corresponds to a specific place; position is where something is.

A virtual electrode template is a computer-generated image of a physical template. A physical electrode template is a device that functions as a jig when temporarily applied to the head of a patient. It carries indicators to show where electrodes are to be attached. A virtual electrode template is shown on a computer monitor or camera viewer as if superimposed on the image of the patient's head as captured by a digital camera and serves as an electrode placement guide for a physical template. Because it is a virtual electrode template, the image does not need to include straps or other physical attributes of a physical template that are needed in a physical template to hold the template in place on the head of the patient, and only uses the relative spatial arrangement of electrode positions associated with the patient's head. The positions may appear to float in fixed spatial relationships based on a mathematical algorithm replicating the prescribed standards for official electrode placement. When the patient moves or turns, the virtual electrodes move or turn as if they were attached to the patient in the same way as physical electrodes would be.

The computer processor may be programmed to locate landmarks on the patient's head that are distinct features, such as the nasion (the bridge of the nose), the inion—which is the area just below the “bump” on the back of the head—, and the left and right pre-auricular points, which are just in front of the left and right ears, respectively, level with the ear canals. The topmost part of the head may also be used as a landmark. There is little confusion in locating these landmarks on the head of any particular person so they may serve as starting points for development of a virtual electrode template. As few as two landmarks may enable the construction of the virtual electrode template such as the nasion and a part of the ear such as the pre-auricle tragus or the entrance to the ear canal. Markers may help to identify a specific point on the auricle, or on a pre-auricle or a post auricle area. For example, markers may be small dot of color applied to the skin that is easily detected because of its contrast with the skin of the patient, and which may be placed, for example, at the nasion and at pre-auricular points.

An example of a simplified system could be based on a mobile device such as a smart phone or pad-type computer programmed for touch-screen capability, including finger gestures and touching for manipulation of the digital image on the camera monitor (viewfinder). Using this method of identifying landmarks or markers, the user faces the patient, centers the digital image of the patient's face and taps the part of the image where the patient's nasion is shown to prompt a software application to ask what feature is begin identified. The user then selects “nasion.” The user walks around the patient, tapping in any order, inion, left pre-auricular area, right pre-auricular area, and the top of the patient's head. The processor of the mobile device, programmed by software, including a mobile device software downloadable application, calculates the positions of the intermediate electrode and adds the calculated positions to the digital image of the patient which has the five positions designated by the user. The processor then generates the virtual electrode template on the monitor of the mobile device in such a way that by moving around the patient, the virtual electrode template appears in the monitor to be on the digital image of the patient's head.

The landmarks are then used by the computer processor in accordance with its programming and user-selectable options to construct the virtual electrode template and superimpose it on the digital image of the patient's head. The virtual electrode template may be one with ten or twenty electrode positions, one electrode position for each of twenty electrodes, or with some other pre-selected and appropriate number and with any configuration of electrodes preferred by the user. The software generates the virtual electrode template showing the positions of virtual electrodes so that a user can see where on the patient's head the electrodes are to be attached. Thus

When a physical electrode is attached to the patient, the digital image on the display, monitor or view finder will show the image of the patient with that electrode and the user can then confirm that electrode is attached in the correct position. The user can also confirm by the declining number of positions on the virtual electrode template where no electrodes are visible how many more electrodes remain and where those electrodes are to be placed.

The user can employ physical tools to assist the computer processor in determining the correct inter-electrode spacing. A tool assists in locating or measurement of the exact dimensions of the head of the patient subject to neurological monitoring. A tool in this case refers to any physical device useful to measure circumference or to determine the location of the crown of the head from, for example, the nasion and inion. For example, such a tool may be two telescoping straps of resilient plastic. The ends of which may be separated when placing one end of one strap on the nasion and the other end of the other strap on the inion. One strap slides with respect to the other until the two, together, reach from nasion to inion. The center point, where the two parts meet, identifies the crown, the mid-point between the respective ends of the straps. Such a tool as that just described may provide input to the digital image identified to the computer processor for locating the crown. A different tool may provide input for the circumference of the patient's head.

A tool may also be used to assist in identifying a landmark. The ears of a patient are easy discerned in a frontal silhouette of a head and they are large landmarks. Ears have multiple features that individually can services as landmarks for comparing one ear to the other. A tool may be attached to the pinnae of the ears that with a vertical member hanging from it with a mark on the vertical member. The vertical position of the marks on the vertical members corresponds to a specific position level with that on the ears, which may be indicated by pointing with the user's finger at the corresponding point on the ears of the digital image of the patient.

The present method may be instantiated as a system that includes a digital camera, a computer processor operable to receive a digital image from the digital camera, and a digital display operatively connected to the computer processor. The processor receives digital images via the camera and displays those images on the monitor of a computer, which can be the viewfinder of a digital camera or screen of a mobile device equipped with a camera. The computer processor is programmed to locate the digital image of the head of the patient using edge detection and face recognition software, and to locate landmarks on the digital image of the patient's head. When landmarks are found, the computer processor generate a virtual electrode template based on those landmarks, and possibly additional locations using markers and mathematical relationships as done currently by technicians, and superimposes the virtual electrode template on the digital image of the patient. The virtual electrode template may then serve as a guide or map of locations on the patient's head where physical electrodes are to be placed. The processor may also be programmed to verify that the locations for the physical electrodes as they are attached, comparing the positions of the electrodes to their locations on the virtual electrode template after refreshing the digital image of the patient's head.

The camera may conveniently be a movie camera so that, when the head of the patient moves, the digital image on the monitor with the virtual electrode template superimposed on it, appear to move together. Markers may include physical objects as described above or thin coatings of readily perceived color. Markers may be temporarily attached or removable with a solvent and may be applied by using a marking pen.

The programmable computer processor and camera may be in the form of a smartphone or tablet computer with a software application installed that enables the use of the built-in camera of the tablet or phone to serve as the display, and capable of user input by touch screen technology techniques. Alternatively, the present system may be incorporated into a pair of glasses with a display in at least one of the eye lenses and a miniature processor supported on the frame.

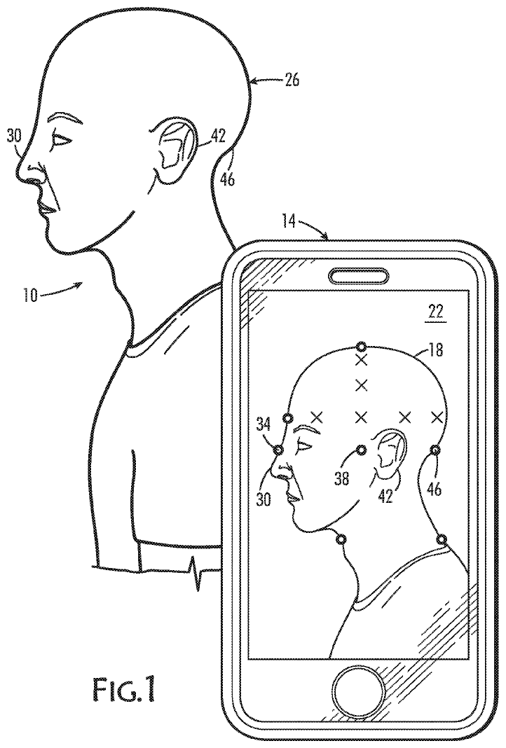

FIGS. 1-3 illustrate a patient 10 and a smart phone 14 with an image 18 of the head of patient 10 in the screen 22 of smart phone 14. A smart phone is a cellular telephone with additional capabilities, such as a camera and screen that shows images passing through the camera lens, and a processor that can be programmed. Smart phone 14 may have other capabilities such as touch screen technology to enable a user to input commands and instructions using a finger placed in contact with screen 22. There are a number of manufacturers of suitable devices, for example, Apple, Inc., which makes a handheld mobile digital electronic device for the sending and receiving of telephone calls, electronic mail, and other digital data, for use as a digital format audio player, and for use as a handheld computer, personal digital assistant, electronic organizer, electronic notepad, and camera, and which is provided under the trademark IPHONE.

For convenience, the term “smart phone” will be used herein to indicate any portable, programmable or programmed device with a camera or video camera, and a monitor capable of showing in real time the image captured by the camera and permitting the user to enter marks on the screen that are temporarily associated with the image then appearing on the screen.

In addition, the present method can be applied using programming modified accordingly for cardiovascular monitoring such as an electrocardiogram that uses electrode placement on the patient's chest, or for other, non-medical monitoring where the arrangement and locations for the electrodes are related to data acquisition.

According to the present method, the user enters the number of electrode locations for the specific type of monitoring or selects the number of electrodes from a menu of options. There are conventions for the numbers of neurological monitoring electrodes and their locations, such as the 10-20 electrode system of the International Federation of Clinical Neurophysiology, and the electrode positions prescribed by these conventions, or user-defined positions and associated position nomenclature may be programmed into the mobile device as choices including one as a default choice.

In use, the user turns the lens of the camera of smart phone 14 toward patient 10 and centers screen 22 on the head 26 of patient 10 so that the user can see image 18 that the camera lens has captured the image of the head 26. The user may then activate software to recognize and focus on specific facial features. The user may touch the image on the screen using a finger or stylus to identify the nasion area of patient 10, which is at the bridge of the nose 30. The user may move with respect to patient 10 to either side of the nasion 34 and indicate in the same manner the pre-auricular point 38, which is just forward of the ear 42 at the level of the ear canal. The user may again rotate with respect to the patient to the back of the head to find and indicate the inion 46, which is just below the small bump on the rearmost part of the scull, or indicate the pre-auricular point 38 on the opposing side of head 26 of patient 10.

These four areas, nasion 34, inion 46, and two pre-auricular points 38, serve as landmarks for constructing the conventional positions over the head 26 of patient 10 where electrodes are customarily placed. Fortunately, as brain mapping continues to reveal locations of the brain that are of interest and importance and the electrode placement choices evolve, these four landmarks are not likely to change and may continue to serve as landmarks even as the preferences for electrode arrangements change.

The software, which has obtained and stored a three-dimensional image of head 26 of patient 10 and has marked at least three of the landmarks, generates a virtual electrode template 50 that appears on screen 22 superimposed on the image of the head 26 of patient 10. If patient 10 moves or rotates his head 26, then virtual electrode template 50 will move or rotate to the same extent and in the same direction as patient 10, thereby tracking the patient's movements.

The identified landmarks are thus used to establish electrode positions 54, that is, where electrodes 58 are to be attached, and may also display virtual electrodes—not only at electrode positions 54 on image 18 of head 26 but also be able to display an electrode number or other designation code associated with that position so that the user can correctly select electrode 58 by number and the locations for those positions 54. The user can then see on screen 22 the positions on image 18 for attaching electrodes 58 to the head 26 of patient 10.

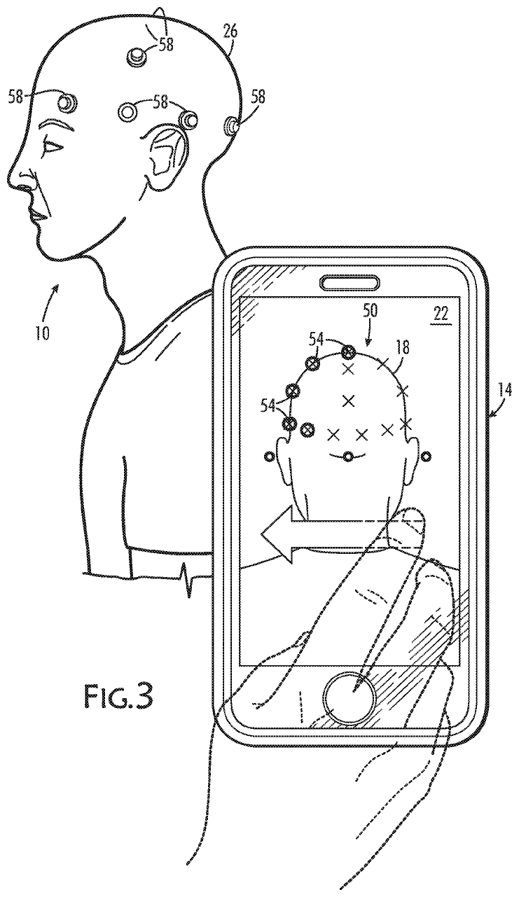

The user moves from location to location, placing electrodes 58 in positions 54 of virtual electrode template 50 the patient's scalp corresponding to those locations. The software can be programmed to verify that the location is the correct location by recognizing the electrodes 58 and their positions 54 and comparing them to the virtual electrode-position information and alerting the user of discrepancies. As best seen in FIG. 3, the user may swipe screen 22 to see an image of virtual electrode template 50 from a different view than that being seen by the camera to verify positions 54 have received an electrode 58.

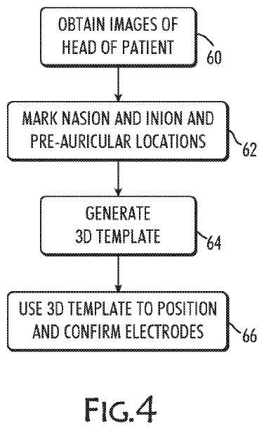

Referring now to FIG. 4, the user may proceed initially at 60, depending on the sophistication of the programming of the camera, by obtaining an image of head 26 of patient 10 using a smart phone 14. Alternatively, the user may at 62 mark the nasion, inion, and pre-auricular points by applying markers or by inputting those positions by touching the image where the markers are to be placed with a cursor, stylus or finger gesture. After the user has identified three of the four landmarks selected from the group consisting of nasion 34, inion 46, and the two pre-auricular points 38 on the image of the patient's head, the software will use that image to generate virtual electrode template 50 at 64 corresponding to the specific geometry of head 26 of patient 10. The user will then, at 66, move and attach electrodes 58 to the positions 54 shown by the three-dimensional version of virtual electrode template 50, using the image 18 on the screen 22 of smart phone 14 to verify correct positioning of each electrode 58 and the completeness of the placements of electrodes 58 on the head of the patient.

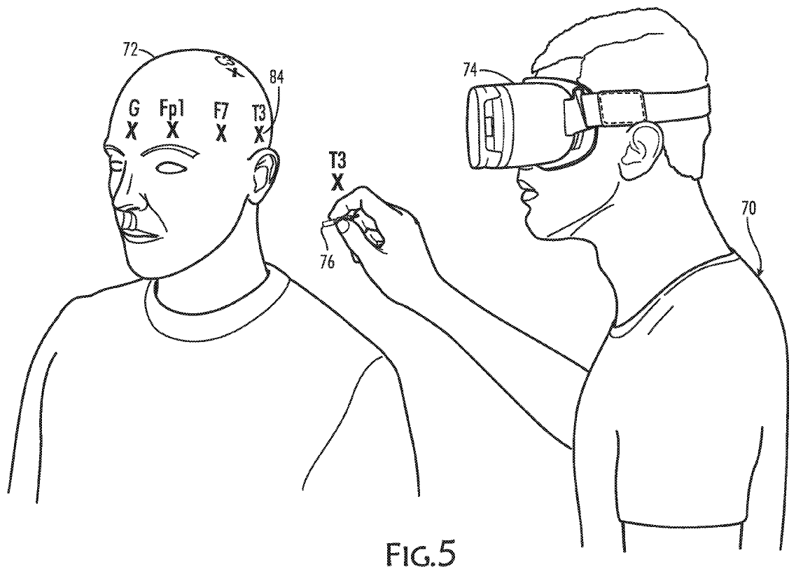

In an alternative aspect of the disclosure, shown in FIG. 5, the user 70 may use a wearable, hands-free, device 74 such as a pair of “computer glasses” or a virtual reality headset. Device 74 locates landmarks such as the nasion, inion, and pre-auricular points based on software that recognizes edges and facial features. Device 74 may include a processor programmed to generate a set of positions for neurological monitoring electrodes that comprises the virtual electrode template. The user, using the virtual electrode template as a guide, may then mark the virtual image of the patient's head 72 with a set of marks, such as a set of letter Xs, so as to guide the user in placing a set of electrodes in the correct positions, such as G, Fp1, F7, T3, and C3, as shown, on the patient's head.

In another, alternative aspect of the disclosure, the present system recognizes the positions of electrodes on the patient's head, and can confirm proper placement by matching the locations of electrodes with their appropriate positions on a virtual template. The electrodes 58 themselves may include a tracking or recognition features, such features as graphic targets, reflective coatings, a locator beacon, radio frequency identification devices (RFIDs) or near field communications (NFC) components, and others, to facilitate the system in recognizing and tracking the electrodes.

The present system may recognize individual electrodes for specific positions. For example, in FIG. 5, the system shows the user that the electrode 76 is intended for the T3 position and is directing the user towards the position 84 on the patient's head. This aspect may be done by using markings on electrode 76 that device 74 recognizes and associates with electrode 76, such as through an RFID chip carried by electrode 76 or other near-field communications technologies. Alternatively, the system may simply recognize an electrode 76 by appearance and direct the user to apply it to a next or most proximate, unoccupied position. The order in which an electrode 76 is applied may be customizable according to the end-user's protocol.

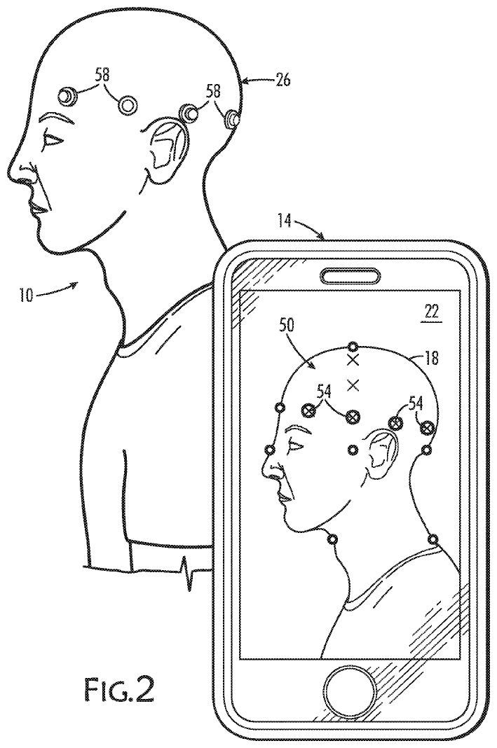

In another aspect of the disclosure, the system may recognize individual electrodes and compare the positions of these electrodes to the positions 54 (shown in FIG. 2) designated by the virtual electrode template in order to confirm the correct placement of the electrodes. For example, the system may recognize generic electrodes, or electrode-shaped objects that are suitable for placement at target positions. Alternatively, accessories may enable the user to adapt generic electrodes for recognition by the system. These accessories may include, for example, stickers, markers, or reflective coatings, and user-applied RFID/NFC devices.

In another aspect, the system may signal to the user that electrode are attached in the positions that correspond to the correct positions and are ready for use. Confirmation of the correct placement of the electrodes may be in real time or by remote monitoring. For example, the user may know by viewing an image on a screen that proper placement of the electrodes is confirmed. Alternatively, the system may send a report of the correct placement for further confirmation by a remote authority or for quality assurance purposes. The report may include captured images or videos of the electrodes as applied to the patient. Once the validity and accuracy of electrode placement is confirmed, the system may then notify the user that monitoring may begin.

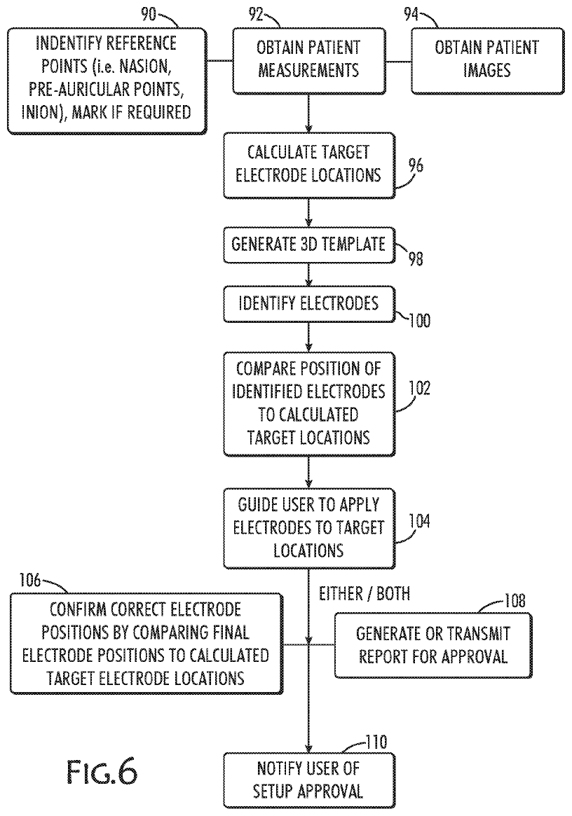

Referring now to FIG. 6, the user of the present system and method may proceed at 90 by identifying reference points on the patient's head. These reference points, metaphorically referred to as landmarks, include the nasion, inion, and auricle-related areas of the ears, such as the pre-auricle tragus and a post-auricle part of the pinna. A digital image of the patient is acquired at 94 using a digital camera with a viewfinder, screen, or monitor, which may also be a movie camera, and which may be a simple mobile device with a camera, processor and memory.

The digital image of the patient and the identified reference points are used to obtain patient measurements at 92 and calculate the set of locations at 96. A three-dimensional virtual electrode template may then be constructed at 98 and superimposed on the digital image of the patient's head.

The electrodes that will be attached to the patient at the positions presented by the virtual electrode template are assembled and may be marked for specific positions or merely identified at 100 so that a specific electrode will be placed at a specific position for quality assurance purposes.

The user attaches an electrode to a location on the head of the patient using the virtual electrode template as a guide. The position of an identified electrode is compared at 102 to the calculated target location specified by the virtual electrode template. The comparison guides the user to apply the electrodes to the target locations at 104. The position of the electrode may be adjusted so that its position matches the location to which it is assigned. When the positions of the electrodes are confirmed to be in the correct locations at 106, a report may be generated and forwarded for approval at 108. On confirmation that the electrodes are in the correct positions, the user may be notified at 110 of setup approval and the user is authorized to commence monitoring.

Those skilled in neurological monitoring under various circumstances will appreciate that many substitutions and variation may be made without departing from the scope of the present disclosure.

1-20. (canceled)

21. A system for use in neurological monitoring, comprising
a mobile device having:
a digital camera,
a monitor operable to display images obtained by said digital camera,
a programmable processor operable to recognize an image of a head of a person when said head is displayed on said monitor and to superimpose a virtual electrode headset having plural virtual electrodes on said image of said head of said person for neurological monitoring; and
a user interface operable to enable adjustment of said virtual electrode headset and positioning of said plural virtual electrodes with respect to said image of said head of said person displayed on said monitor, said user interface enabling correction of positions of said virtual electrodes as displayed on said image of said head and to thereby generate said virtual electrode headset, said virtual electrode headset as displayed conforming to said image of said head of said person and rotating therewith as displayed and thereby guiding a user in applying electrodes to said locations on said head of said person whose image is displayed on said monitor.

22. The system of claim 21, said mobile device having a smart phone.

23. The system of claim 21, said mobile device having a pad computer.

24. The system of claim 21, said programmable processor being operable to recognize an orientation of said head of said person as said person appears on said monitor.

25. The system of claim 24, said head having landmarks and said programmable processor is programmed to recognize said landmarks and generate a virtual electrode template therefrom.

26. A method for neurological monitoring, comprising:
(a) capturing a digital image of a head of a person, said digital image having physical features appearing on said head of said person using a mobile device, having a processor, a memory, a digital camera, a monitor, and a user interface;
(b) displaying said digital image on said monitor of said mobile device;
(c) identifying said physical features in said digital image using said user interface;
(d) generating using said processor a set of locations on said digital image for a virtual electrode template;
(e) displaying said virtual electrode template on said monitor of said digital camera; and
(f) attaching electrodes to said head of said person corresponding to said set of electrode locations using said virtual electrode template as a guide.

27. The method of claim 26, further comprising marking physical features of said head of said person on said monitor to facilitate identification of said physical features in said digital image on said monitor.

28. The method of claim 26, further comprising calculating distances between physical features using said processor using said digital image.

29. The method of claim 26, further comprising programming said processor to add image locations on said monitor as said head of said person turns.

30. The method of claim 26, further comprising moving said virtual electrode template, using said user interface, when said virtual electrode is not in a virtual electrode location.

31. The method of claim 26, further comprising indicating when an electrode placed on said head of said person is in a virtual electrode location.

32. The method of claim 26, further comprising superimposing said virtual electrode template on said digital image with said image features aligned with said physical features.

33. The method of claim 32, further comprising generating additional digital neurological monitoring locations by said processor as said head turns and displaying said additional digital neurological monitoring locations as part of said virtual digital template as said head turns.

34. The method of claim 26, said method further comprising:
(a) refreshing said digital image on said monitor to show an electrode in a position at an electrode location on said head of said patient;
(b) comparing said position of said electrode in said digital image to said electrode location in said virtual electrode template; and
(c) determining whether said position of said electrode coincides with said electrode location of said set of electrode locations of said virtual electrode template.

35. The method of claim 34, further comprising adjusting said position of said electrode of said set of electrodes when a location to which said electrode of said set of electrodes is attached does not correspond to said electrode position of said set of electrode positions.

36. The method of claim 35, further comprises embedding said electrode into said head of said patient.

37. A method, comprising:
(a) capturing a digital image of a head of a patient using a digital camera, said head of said patient having a feature, and said digital camera having a monitor and a processor;
(b) displaying said digital image on said monitor;
(c) identifying said feature on said head of said patient in said digital image;
(d) designating a first location for positioning an electrode on said head of said patient relative to said feature on said head of said patient;
(e) generating a virtual electrode headset based said first location, said virtual electrode headset defining electrode locations for a set of electrodes for neurological monitoring of said patient; and
(f) positioning a set of electrodes on said head of said patient in accordance with said electrode locations said virtual electrode headset.

38. The method of claim 37, said method further comprising the steps of:
(a) comparing positions of said set of electrodes on said head of said patient with said virtual set of electrodes; and
(b) adjusting said positions of said electrodes to conform said positions of said electrodes to said locations for electrodes of said virtual set of electrodes.

39. The method of claim 37, further comprising the step of attaching said electrodes to said head of said patient.
